(19) United States
(12) Patent Application Publication
(10) Pub. No.: US 2021/0027892 A1
(43) Pub. Date: Jan. 28, 2021

(54) SYSTEM AND METHOD FOR OUTPUTTING GROUPS OF VECTORIZED TEMPORAL RECORDS

(71) Applicant: Knowtions Research Inc., Toronto (CA)

(72) Inventors: Anthony LEE, Toronto (CA); Alexandre TOMBERG, Toronto (CA); Kaveh HASSANI, Toronto (CA)

(21) Appl. No.: 17/044,953

(22) PCT Filed: Jun. 29, 2018
(86) PCT No.: PCT/IB2018/054863

§ 371 Date: Oct. 2, 2020

Related U.S. Application Data
(60) Provisional application No. 62/652,482, filed on Apr. 4, 2018.

(51) Int. Cl. G16H 50/20; G16H 50/70; G06N 3/08; G06F 16/90

(52) U.S. Cl. CPC G16H 50/20; G06F 16/90; G06N 3/08; G16H 50/70

(57) ABSTRACT

Systems and methods of identifying groups of actors based on temporal records and outputting groups of vectorized temporal records are provided. A prediction engine receives a vector representing a temporal record comprising an event having a timestamp. The prediction engine generates a probability score of a future event associated with the temporal record. The vector is grouped with one or more additional vectors to form a group, the grouping based on the temporal record and the probability score. The group is then output.

### CROSS-REFERENCE TO RELATED APPLICATIONS

This application claims priority to U.S. App. Ser. No. 62/652,482, filed Apr. 4, 2018, the entirety of which is incorporated herein by reference.

### FIELD

The specification relates generally to a method and system for outputting groups of vectorized temporal records, and in particular, outputting groups of vectorized temporal records based on actual and predicted events.

### BACKGROUND

When events occur, data pertaining to the event is generated. The data may represent when the event occurred, what the event was, why the event occurred, and so on. The data may also represent actor data, such as people, parties, or objects which experienced the event, and information associated with the actor. The data generated from an event therefore can include many data points and can be highly dimensional. As more events occur over time, the dimensionality of the sequence of events can increase. For example, over their lifetimes, people generate healthcare data for each medical episode they experience.

### SUMMARY

An aspect of the specification provides a method for outputting groups of vectorized temporal records. The method includes receiving, at a prediction engine, a vector representing a temporal record comprising an event having a timestamp. The method further includes generating, at the prediction engine, a probability score of a future event associated with the temporal record. The method further includes grouping the vector with one or more additional vectors to form a group, the grouping based on the temporal record and the probability score. The method further includes outputting the group.

According to an implementation, the method further includes receiving, at the prediction engine, one or more further vectors representing corresponding further temporal records, each further temporal record comprising one or more events having corresponding timestamps.

According to an implementation, the probability score is generated based on the temporal record and the one or more further temporal records.

According to an implementation, the prediction engine comprises a neural network trained using the one or more further temporal records.

According to an implementation, the method further comprises generating, at a vectorization engine, the vector based on the temporal record, the temporal record associated with an actor.

According to an implementation, the method further comprises updating the vector based on an updated temporal record comprising the event having the timestamp and a further event having a further timestamp.

According to an implementation, the method further comprises detecting a change in the temporal record, and updating the vector to reflect the change in the temporal record.

According to an implementation, the method further comprises generating, at the prediction engine, further probability scores of a plurality of further future events associated with the temporal record.

According to an implementation, the grouping is based on the temporal record, the probability score and the further probability scores.

According to an implementation, outputting the group comprises generating a visual representation of the group.

According to an implementation, the method further comprises comparing an actual event associated with the temporal record with a predicted event, the predicted event comprising the future event when the probability score is above a threshold score. The method further comprises generating a notification if an inconsistency is detected between the actual event and the predicted event.

An aspect of the specification provides a system for identifying groups of actors based on temporal records. The system comprises a prediction engine including a processor in communication with a computer-readable memory. The prediction engine receives a vector representing a temporal record comprising an event having a timestamp. The prediction engine further generates a probability score of a future event associated with the temporal record. The prediction engine further outputs a group comprising the vector and one or more additional vectors grouped together based on the temporal record and the probability score.

According to an implementation, the prediction engine is further configured to group the vector and the one or more additional vectors based on the temporal record and the probability score.

According to an implementation, the prediction engine is further configured to receive one or more further vectors representing corresponding further temporal records, each further temporal record comprising one or more events having corresponding timestamps.

According to an implementation, the prediction engine is configured to generate the probability score based on the temporal record and the one or more further temporal records.

According to an implementation, the prediction engine comprises a neural network trained using the one or more further temporal records.

According to an implementation, the system further comprises a vectorization engine configured to generate the vector based on the temporal record, the temporal record associated with an actor.

According to an implementation, the vectorization engine is further configured to update the vector based on an updated temporal record comprising the event having the timestamp and a further event having a further timestamp.

According to an implementation, the vectorization engine is further configured to detect a change in the temporal record and update the vector to reflect the change in the temporal record.

According to an implementation, the prediction engine is further configured to generate probability scores of a plurality of future events associated with the temporal record.

According to an implementation, the prediction engine is configured to group the vector and the one or more additional vectors based on the temporal record, the probability score, and the further probability scores.

According to an implementation, the prediction engine is configured to generate a visual representation of the group.

According to an implementation, the prediction engine is further configured to compare an actual event associated with the temporal record with a predicted event, the predicted event comprising the future event when the probability score is above a threshold score; and generating a notification if an inconsistency is detected between the actual event and the predicted event.

An aspect of the specification provides a computer-readable storage medium having recorded thereon instructions executable by a processor of a prediction engine. The instructions cause the processor to receive a vector representing a temporal record comprising an event having a timestamp, the temporal record associated with an actor. The instructions further cause the processor to generate a probability score of a future event associated with the temporal record. The instructions further cause the processor to group the vector with one or more additional vectors to form a group, the grouping based on the temporal record and the probability score. The instructions further cause the processor to output the group.

### BRIEF DESCRIPTIONS OF THE DRAWINGS

Implementations are described with reference to the following figures, in which:

FIG. 1 depicts an example system for identifying groups of actors based on temporal records;

FIG. 2 depicts certain internal components of a prediction engine and a vectorization engine of the system of FIG. 1;

FIG. 3 depicts a method of identifying groups of actors based on temporal records;

FIG. 4 depicts a method for preprocessing prior to the method of FIG. 3; and

FIG. 5 depicts a schematic diagram an example iteration in the system of FIG. 1.

### DETAILED DESCRIPTION

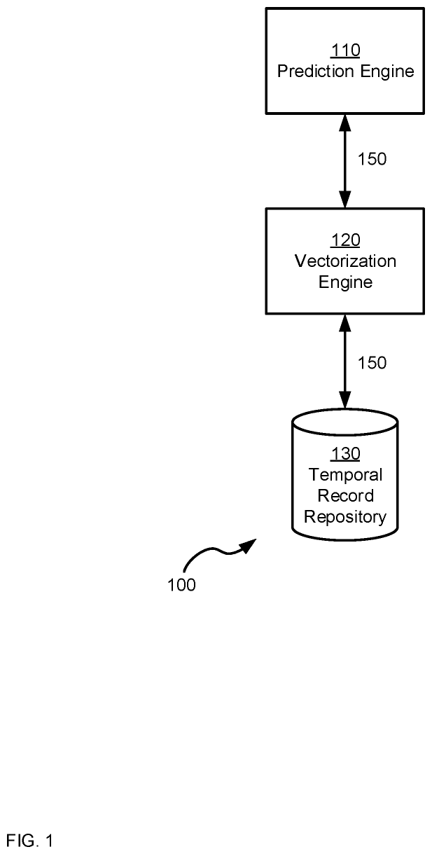

FIG. 1 depicts an example system 100 for identifying groups of actors based on temporal records. The system 100 includes a prediction engine 110, a vectorization engine 120 and a temporal record repository 130. In some implementations, the system 100 may further include a client terminal (not shown) for receiving and displaying outputs from the prediction engine 110. The client terminal may be a computing device such as a desktop computer, a laptop computer, a tablet computer, a mobile phone, or other suitable device. The prediction engine 110 and the vectorization engine 120 may comprise servers; examples of certain internal components of the prediction engine 110 and the vectorization engine 120 will be discussed below.

The components of the system 100 are in communication via communication links 150. In particular, the prediction engine 110 is in communication with the vectorization engine 120. In some implementations the prediction engine 110 is further in communication with the client terminal. The vectorization engine 120 in turn, is in communication with the temporal record repository 130. The communication links 150 may include internet protocol (IP) networks, such as intranet, a local-area network, a wide-area network, a virtual private network (VPN), a Wi-Fi network, the internet, combinations of such, and the like. The communication links 150 may be direct links, or links that traverse one or more networks, including both local and wide-area networks.

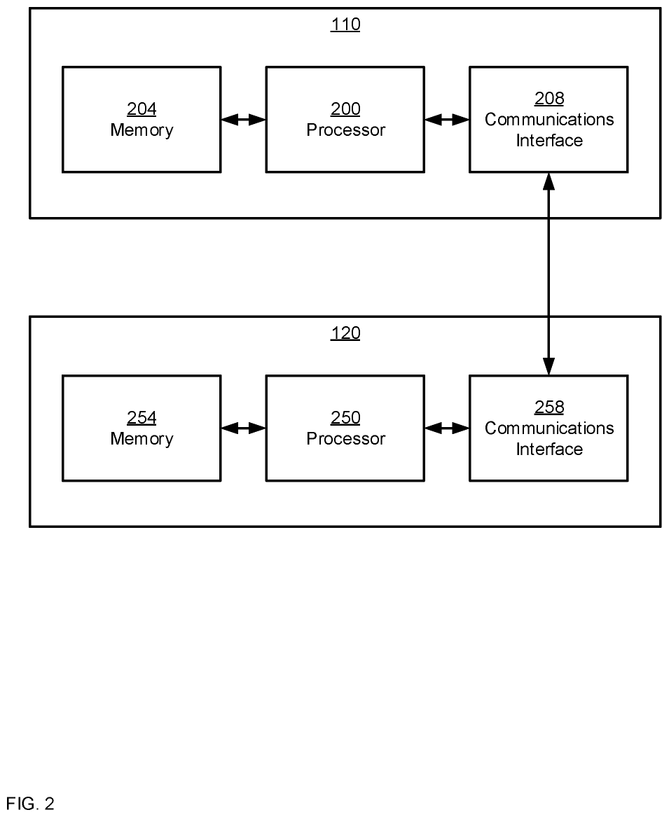

FIG. 2 depicts certain components of the prediction engine 110 and the vectorization engine 120. The prediction engine 110 includes a processor 200. The processor 200 may include a central-processing unit (CPU), a microcontroller, a microprocessor, a processing core, a field-programmable gate array (FPGA), a graphics processing unit (GPU) or similar. The processor 200 may include multiple cooperating processors. In some implementations, the prediction engine 110 may be implemented on a cloud computing network, and the functionality described herein may be implemented as a cloud-based service. In some implementations, the processor 200 may be configured to implement a neural network to execute the various functions described herein. The processor 200 can comprise any suitable neural network system or combination of neural network systems, such as recurrent neural networks, long-short term memory neural networks, and the like. The processor 200 may cooperate with a non-transitory computer-readable storage medium such as a memory 204 to execute instructions to realize the functionality discussed herein.

The memory 204 may include a combination of volatile (e.g. Random Access Memory or RAM) and non-volatile memory (e.g. read only memory or ROM, Electrically Erasable Programmable Read Only Memory or EEPROM, flash memory). All or some of the memory 204 may be integrated with the processor 200. The memory 204 stores computer-readable instructions for execution by the processor 200. In some implementations, the memory 204 is configured to store information related to actors, temporal records and/or vectors. For example, the memory 204 may store generated probability scores and groups for further processing.

The prediction engine 110 also includes a communications interface 208 interconnected with the processor 200. The communications interface 208 can include suitable hardware (e.g. transmitters, receivers, network interface controllers and the like) allowing the prediction engine 110 to communicate with other computing devices, such as the vectorization engine 120 and the client terminal via the communication links 150. The specific components of the communications interface 208 are selected based on the type of network or other links over which the prediction engine 110 communicates.

The vectorization engine 120 can be similar to the prediction engine 110. Hence, the vectorization engine 120 includes a processor 250. The processor 250 may include a central-processing unit (CPU), a microcontroller, a microprocessor, a processing core, a field-programmable gate array (FPGA), a graphics processing unit (GPU) or similar. The processor 250 may include multiple cooperating processors. In some implementations, the vectorization engine 120 may be implemented on a cloud computing network, and the functionality described herein may be implemented as a cloud-based service. In some implementations, the processor 250 may be configured to implement a neural network to execute the various functions described herein. The processor 250 can comprise any suitable neural network system or combination of neural network systems, such as recurrent neural networks, long-short term memory neural networks, and the like. The processor 250 is configured to cooperate with a non-transitory computer-readable medium, such as a memory 254, to execute instructions to realize the functionality described herein. In some implementations, the memory 254 is further configured to store information relating to actors, temporal records and vectors. For example, the memory 254 may store associations between vectors and actors for further processing. The vectorization engine also includes a communications interface 258 configured to allow the vectorization engine 120 to communicate with other computing devices, such as the prediction engine 110 and the temporal record repository 130, via the communication links 150.

In some implementations, the vectorization engine 120 may be distinct from the prediction engine 110, as is shown in the present example. In other implementations, the vectorization engine 120 may be integrated with the prediction engine 110.

The temporal record repository 130 stores temporal records. Each temporal record is associated with an actor and includes one or more events having respective timestamps. For example, the temporal record may include a first event having a first timestamp, a second event having a second timestamp, and so on, including an n-th event having an n-th timestamp. Accordingly, the temporal record represents an event or a series of events occurring over a duration of time. In some implementations, the temporal record may further include one or more codes associated with each event, the codes representing further information associated with the event. For example, the codes may be alpha-numeric strings, may have a predetermined length or the like. For example, the codes may be medical codes as defined by the World Health Organization (WHO) in the International Statistical Classification of Diseases and Related Health Problems (ICD). In some implementations, the temporal record may further include, for each event, actor information defining actor characteristics at the time of the event.

In some implementations, the temporal record repository 130 may be integrated with the with the memory 204 of the prediction engine 110, or with the memory 254 of the vectorization engine 120.

For example, the temporal record may represent a history of medical episodes experienced by the actor, with the timestamp representing the date of occurrence of the episode. Accordingly, the temporal record may further include codes representing test result information, diagnosis information, symptom information, treatment information and the like associated with each medical episode. The temporal record may further include, for each medical episode, variable actor information specific to the date of occurrence of the episode (i.e. the timestamp), such as age, occupation, and the like. The variable actor information are actor characteristics which may change over time (age, occupation, etc.). Accordingly, the actor information which is accurate at the time or date of occurrence of the episode is stored in association with the episode. The actor may be a patient, a doctor, or other party involved in a series of medical episodes.

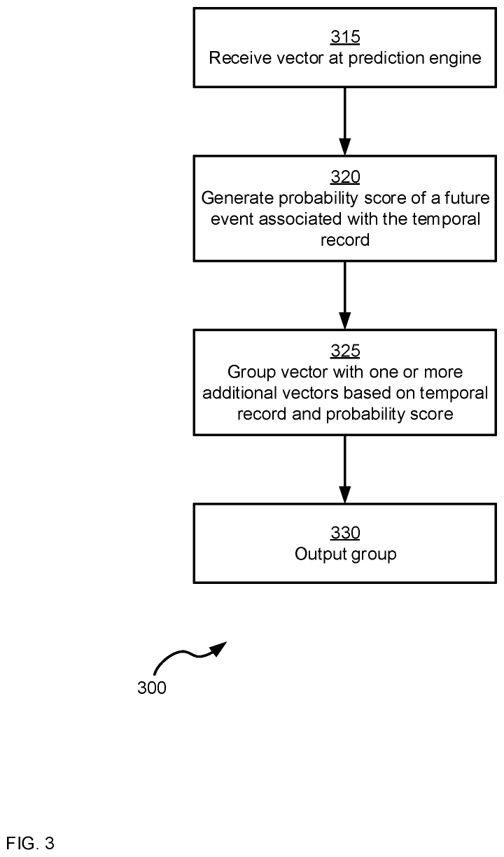

Turning now to FIG. 3, a flowchart depicting an example method 300 is shown. The method 300 can be used for identifying groups of actors based on temporal records. For ease of description, the method 300 will be described below in connection with its performance in the system 100 as illustrated in FIG. 1. The blocks of the method 300 can be performed by the processor 200 and/or the processor 250. In some examples, the method 300 can also be implemented or performed using a suitable system other than system 100.

At block 315, the prediction engine 110, and particularly the processor 200, receives an input vector representing a temporal record. For example, the prediction engine 110 may receive the input vector from the vectorization engine 120. In some implementations, the prediction engine 110 may receive one or more further vectors in addition to the input vector, the one or more further vectors representing corresponding further temporal records. For example, the prediction engine 110 may also receive the one or more further vectors from the vectorization engine 120. In other examples, the one or more further vectors may have been previously received at the prediction engine 110 and stored in the memory 204. Hence, the one or more further vectors may be retrieved from the memory 204 by the processor 200 for processing the input vector, as will be described further below. Accordingly, upon receipt of the input vector, the prediction engine 110 may be configured to store the input vector in the memory 204 for further processing.

At block 320, the prediction engine 110, and in particular the processor 200, generates a probability score of a future event associated with the temporal record. Specifically, the probability score indicates the probability of the future event occurring given the events of the temporal record. In some implementations, the probability score is a probability value (e.g. a decimal value in the range of 0 to 1, or a percentage between 0 and 100%), while in other implementations, the probability score is an indicator based on the probability (e.g. low/medium/high). In some implementations, the probability score is generated based on the temporal record and the one or more further events. That is, the probability of the future event may be calculated based on relationships between the event information of the one or more further temporal records and the temporal record of interest. For example, the relationships between event information may be similarities in relative time intervals of occurrence of the episode (e.g. events occurring when the actor is between 20-30 years of age), similarities between event type, relationships between the actors associated with the temporal records, and the like. The further temporal records may be temporal records received and stored during prior iterations of the method 300. In other examples, the further temporal records may be received as further vectors concurrently with the input vector at the block 315.

For example, the temporal record may represent the medical history of a patient. Events in the temporal record may include medical episodes such as chest pain, fainting, heart palpitations and the like. The temporal record may further include additional information about the episodes such as high blood pressure, shortness of breath, diagnosis of heart disease, test results of an electrocardiogram (ECG) test and the like. Accordingly, the processor 200 may generate a probability score of the patient having a heart attack (i.e. the future event). The processor 200 may, for example, generate the probability score based on the prior medical episodes of the patient, and based on the temporal records associated with other patients having any one or a combination of: similar high blood pressure, shortness of breath, diagnosis of heart disease, similar ECG test results and so on.

Moreover, in some examples, the processor 200 may also consider events having similar variable actor information, such as patient age at the time of occurrence of the medical episode. Accordingly, the probability score may be generated based on a comparison between events occurring at a relatively similar time frame in the patient's life, rather than the objective timestamps associated with the events. For example, the probability score may be based on temporal records associated with patients having a diagnosis of heart disease between 40 and 50 years of age (i.e. based on the variable actor information). In further examples, the processor 200 may consider the relations between temporal records. For example, the probability score of a future medical episode of a patient may be correlated to the experience and/or success rate of the doctor with whom the patient has a doctor-patient relationship.

In some implementations, where the processor 200 implements a neural network, the neural network may be trained using the further temporal records. That is, the neural network may be trained for pattern recognition and probability score generation according to the further temporal records, and in consideration of events having similar event information.

In some implementations, the prediction engine 110, and in particular the processor 200, is configured to generate further probability scores of a plurality of further future events associated with the temporal record. In other words, the processor 200 generates a set of probability scores, each associated with a different possible future event, or sequence of future events. In some implementations, the processor 200 may set a minimum threshold score for the probability scores to be included in the set. Hence the set of probability scores corresponds with a set of events having a predetermined minimum probability. In some examples, each probability score of the set may be generated, independently of each other, based on the temporal records and the further temporal records. In other examples, the probability scores may be generated based on the temporal records, the further temporal records, and other generated probability scores.

At block 325, the input vector is grouped with one or more additional vectors to form a group. Specifically, the grouping is based on the temporal record and the probability score.

In some implementations, the grouping can comprise the following: vectorization engine 120 may receive the probability score of the future event associated with the temporal record. The vectorization engine 120 may generate a modified input vector associated with the actor, the modified input vector generated based on the temporal record and the probability score, as will be described further below. In such implementations, the modified input vector may be grouped with one or more additional vectors. For example, the additional vectors may similarly be vectors modified with corresponding probability scores. Accordingly, the modified input vectors may be grouped with one or more modified additional vectors according to clustering algorithms, such as hierarchical density-based spatial clustering of applications with noise (HDBSCAN), principal component analysis (PCA), single-linkage clustering, or the like.

In other implementations, the grouping may be based on a direct comparison between the events of the input temporal record and events of the additional temporal records represented by the additional vectors, as well as a comparison between the probability score of the future event associated with the input temporal record and probability scores of future events associated with the additional temporal records. In some implementations, the grouping may be based on a comparison between the probability score of the future event associated with the input temporal record and the events of the additional temporal records. Moreover, in some implementations, the grouping may also be based on the probability scores of the further future events. Other grouping methodologies are also contemplated.

In some implementations, the grouping may be performed by the prediction engine 110. In other implementations, another computing engine, such as a grouping engine, or a processer associated with the client terminal, may be configured to group the input vector or modified input vector with one or more additional vectors based on the temporal record and the probability scores.

At block 330, the group is output. Outputting can comprise sending the group or group information to a display, a printer, another device or network. In some examples, outputting the group can comprise sending the group information to and/or making the group information available to other systems, networks, or human operators. In some implementations, the output may be a visual representation of the group. For example, the modified vectors may be plotted and displayed in a distinct color from non-grouped additional vectors. In other examples, the output may be a listing of the actors and/or temporal records associated with the grouped vectors. In further examples, the temporal record of each actor in the group may be visually marked as being part of the group.

In other implementations, the system 100 may further be configured to detect inconsistencies in the temporal record. In particular, an actual event associated with the temporal record is compared to a predicted event for the temporal record, where the predicted event is predicted to occur over the same timeframe as the actual event. The predicted event may be a future event having a probability score above a given threshold score. If an inconsistency is detected between the actual event and the predicted event, a notification is generated to alert a user of the inconsistency. For example, the predicted event may be a future event having a probability score below a threshold score (i.e. suggesting that the future event is unlikely to happen). Accordingly, an inconsistency may be detected if the event is realized in the temporal record.

In other implementations, a notification may be generated where the inconsistency is related to the actor being moved from one group to another group based on changes to the temporal record associated with the actor. For example, as the frequency and severity of a patient's medical episodes increase over the patient's lifetime, the patient may move from a designated healthy group to a group designated as higher risk for future medical episodes. Other outputs of the group are also contemplated.

In some implementations, the output is produced by the prediction engine 110. In other implementations, the output is produced by another computing device, such as the processor associated with the client terminal.

In some implementations, a non-transitory computer-readable storage medium (CRSM) may be provided independently of the system 100. The CRSM may have recorded thereon instructions executable by a processor. The CRSM may comprise an electronic, magnetic, optical or other physical storage device that stores executable instructions. The instructions may comprise instructions to cause the processor to receive a vector representing a temporal record comprising an event having a timestamp, the temporal record associated with an actor. The instructions may further cause the processor to generate a probability score of a future event associated with the temporal record. The instructions may further cause the processor to group the vector with one or more additional vectors to form a group, the grouping based on the temporal record and the probability score. The instructions may further cause the processor to output the group.

Moreover, the methods, systems, and CRSMs described herein may include the features and/or perform the functions described herein in association with one or a combination of the other methods, systems, and CRSMs described herein.

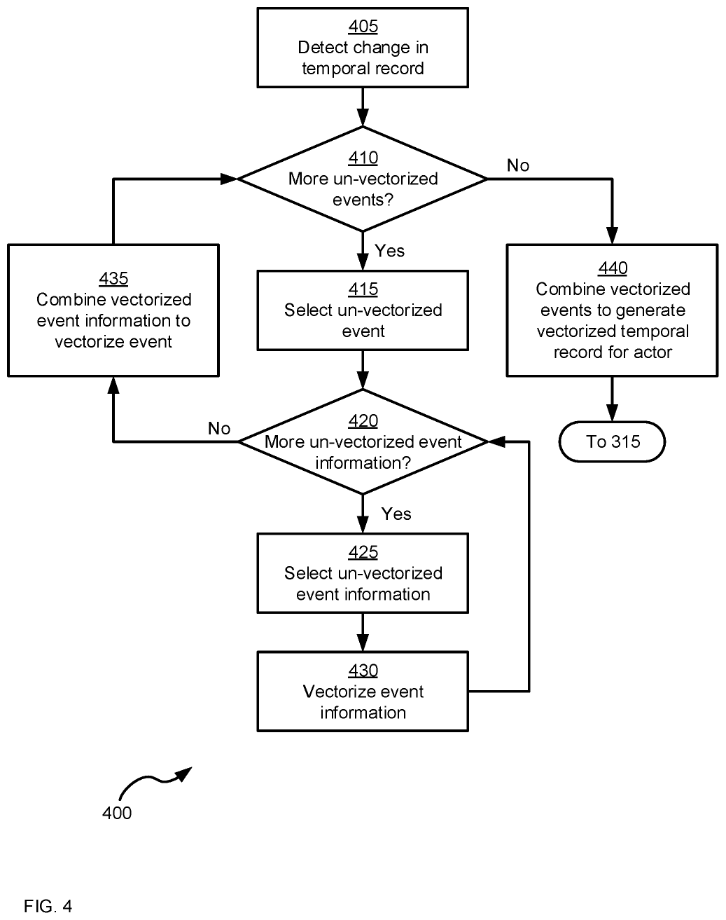

Turning now to FIG. 4, a flowchart depicting an example method 400 is shown. The method 400 may be used as a preprocessing operation prior to the method 300. For ease of description, the method 400 will be described below in connection with its performance in the system 100 as illustrated in FIG. 1. The blocks of the method 400 can be performed by the processor 200 and/or the processor 250. In some examples, the method 400 can also be implemented or performed using a suitable system other than the system 100.

At block 405, the vectorization engine 120 detects a change in a temporal record. In some examples, the change may be a new temporal record added to the repository 130, while in other examples, the change may be an update to an existing temporal record in the repository 130. For example, the vectorization engine 120 may be configured to monitor the temporal record repository 130 to detect changes in the temporal records. Specifically, the vectorization engine 120 may detect updates to temporal records or additions of new temporal records. In another example, the repository 130 may be configured to communicate changes, such as updates or additions, in temporal records to the vectorization engine 120 via the communication links 150.

In some implementations, the change may be a new temporal record added to the repository 130. For example, the new temporal record may be associated with an actor not currently associated with any other temporal record stored in the repository 130. In another example, a new temporal record may be generated in association with a known actor. The new temporal record therefore has at least one event and an associated timestamp. In other implementations, the change may be an update to an existing temporal record. That is, the existing temporal record associated with a known actor is updated, or example to add a new event having a new timestamp to the existing temporal record. In other examples, the update may be a change in the codes, or other information associated with an event of the existing temporal record.

For example, where the temporal records represent the history of medical episodes, a doctor may acquire a new patient and treat the patient for chest pain. Accordingly, the temporal record of the doctor may be updated with information about the patient's chest pain episode, including the date of the appointment, the treatment plan prescribed (e.g. medication, prescribed medical tests, etc.), and any further diagnosis, treatment, or other information stored in the temporal record. Further, a new temporal record for the new patient may be generated, and may similarly have the chest pain episode, including the date of the appointment, the symptoms experienced (e.g. chest pain, nausea, etc.), the treatment plan received, and any further diagnosis, treatment, or other information stored in the temporal record. Hence, the vectorization may detect multiple changes—a new temporal record added for the new patient, and an update to the temporal record of the doctor. At a later date, the test results for the patient may be received, resulting in a diagnosis of heart disease. Accordingly, the temporal records for the patient and doctor may be updated. Specifically, the temporal record of the doctor may be updated with the test result information (e.g. results from X-ray, electrocardiogram tests, etc.), a diagnosis code indicating the heart disease diagnosis, and an updated treatment plan. Similarly, the temporal record of the patient may also be updated with the test result information, the diagnosis code indicating the heart disease diagnosis, and the updated treatment plan.

Upon detecting a change in a temporal record, the vectorization engine 120 is configured to proceed to block 410. At block 410, the vectorization engine 120, and particularly the processor 250, determines whether the temporal record includes events which have not yet been vectorized. If there are un-vectorized events, the vectorization engine 120 is configured to proceed to block 415.

At block 415, the processor 250 selects an un-vectorized event and proceeds to block 420. For example, the processor 250 may select a particular medical episode of the temporal record.

At block 420, the processor 250 determines whether the un-vectorized event includes event information which has not yet been vectorized. If there is un-vectorized event information, the processor 250 proceeds to block 425.

At block 425, the processor 250 selects a type of un-vectorized event information and proceeds to block 430. For example, types of un-vectorized event information may include variable actor information, such as age at the time of occurrence of the event, or medical codes representing diagnosis information, test results, and the like.

At block 430, the processor 250 vectorizes the event information to generate an event information vector.

In some implementations, the event information vector may be generated by implementing co-occurrence matrices from possible event information. For example, a co-occurrence matrix may be implemented to determine semantic similarity of event information such as the medical codes, or doctor's notes associated with the medical episode. Specifically, the co-occurrence matrix may include rows and columns corresponding to codes in the coding terminology (e.g. medical codes). The matrix values may then be counts of the number of times two codes co-occur. The processor 250 may then generate a sub-matrix from the larger co-occurrence matrix and assign a vector for each row and each column of the sub-matrix, according to a predetermined target dimensionality. The processor 250 may continue iteratively to compute a final vector for each code. In particular, the co-occurrence matrix has a high degree of dimensionality, which is iteratively reduced until a target dimensionality is reached for particular codes.

In other implementations, the event information vector may be generated by generating one-hot vectors. In particular, one-hot encoding binarizes categorical data in terms of 1 (true) or 0 (false), where the only non-zero component is the corresponding event information which is being vectorized (e.g. actor is 20-29 years of age).

The event information vector may then be stored, for example, in the memory 254 for further processing. The processor 250 is then configured to return to block 420 to determine whether further un-vectorized event information exists.

If at block 420, there is no un-vectorized event information for the event selected at block 415, the processor 250 proceeds to 435. At block 435, the processor 250 vectorizes the event to generate an event vector. For example, the processor 250 may vectorize the event by concatenating or otherwise combining the event information vectors. In particular, the processor 250 may further reduce the dimensionality of the multiple event information vectors to generate the event vector, while representing and encoding the event information. The event vector may then be stored, for example, in the memory 254, for further processing. The processor 250 is then configured to return to block 410 to determine whether further un-vectorized events exist.

If at block 410, there are no un-vectorized events for the temporal record, the processor 250 proceeds to block 440. At block 440, the processor 250 vectorizes the temporal record to generate a temporal record vector. For example, the processor 250 may vectorize the event by concatenating or otherwise combining the event vectors. Accordingly, the temporal record vector represents and encodes the information of the temporal record while having a reduced dimensionality as compared to the temporal record.

In some implementations, the processor 250 may further concatenate or otherwise combine the temporal record vector with static actor information, such as date of birth, and the like to generate an actor vector. The static actor information may be represented, for example, as another one-hot encoded vector.

In other implementations, the processor 250 may further concatenate or otherwise combine the temporal record vector with a probability score of a future event to generate a modified temporal record vector or a modified actor vector generated at block 320 of the method 300. The probability score of the future event may be represented, for example as a one-hot encoded vector. The modified actor vector therefore encodes event information associated with actual events from the temporal record, as well as predictive information based on the probability score of the future event.

Where the temporal record is a new temporal record associated with a new actor, the vectorization engine 120 generates a new input vector representing the new temporal record and associated with the new actor. The vectorization engine 120 may store the generated vector in association with the actor in the memory 254 for future processing. Where the temporal record is an existing temporal record associated with a known actor and having a change or an update, the vectorization engine 120 updates the vector to reflect the change in the temporal record. In particular, the vectorization engine 120 may generate an updated vector based on the updated temporal record, and store the updated vector in association with the actor for future processing.

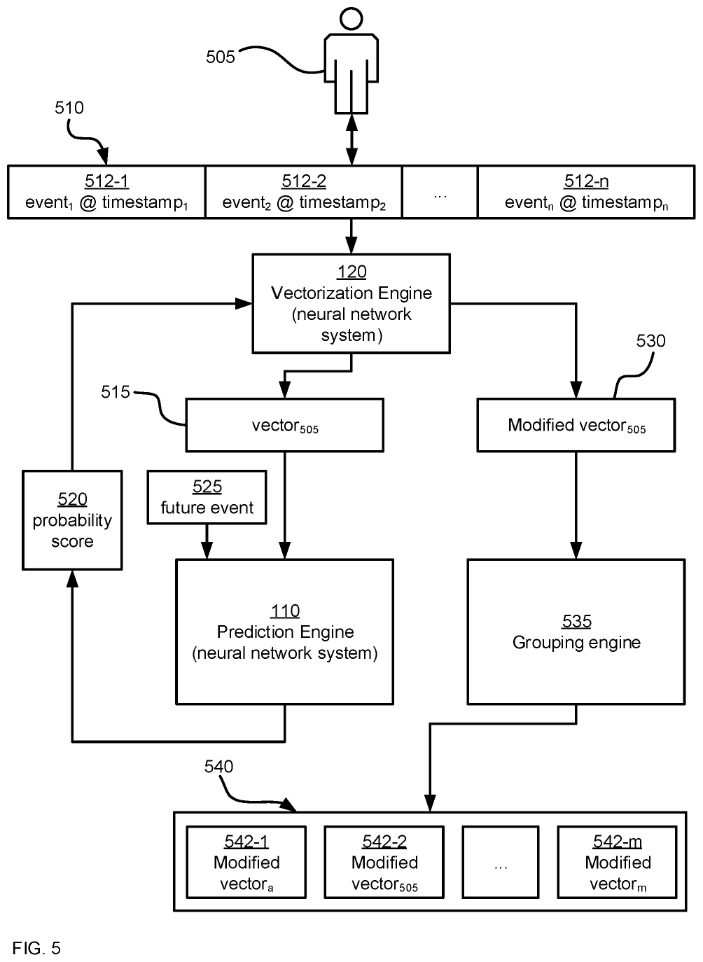

Turning now to FIG. 5, a schematic diagram of an example scenario of identifying groups of actors based on temporal records is depicted. For ease of description, the example scenario will be described below in connection with the method 300 as performed by the system 100.

FIG. 5 depicts an actor 505 associated with a temporal record 510. For example, the actor 505 may be a patient, and the temporal record 510 may be a series of medical episodes that the patient has experienced. In particular, the temporal record 510 includes a plurality of events 512-1, 512-2, . . . , 512-n (collectively referred to as events 512 and generically referred to as an event 512) having corresponding timestamps. The events 512 may further be associated with medical codes, such as diagnosis codes, treatment codes, lab test data, and the like. The events 512 may further be associated with variable actor information specific to the event 512. The temporal record 510 may be stored in the temporal record repository 130 (shown in FIG. 1). In some examples, the repository 130 may store medical insurance related data. Accordingly, the temporal record 510 may further include, for each event 512, claim approval information, claim amounts, insurance underwriter identifier and similar information.

In the present example, the temporal record 510 may have been updated to reflect a change in the temporal record, for example, by updating a claim amount of an event 512. Accordingly, at block 405 (shown in FIG. 4), the vectorization engine 120 may receive the temporal record 510 and update a vector 515 associated with the actor 505 to reflect the changes in the temporal record 510 in accordance with the method 400. In particular, the vector 515 encodes the information of the events 512, including the changes to the temporal record 510.

The vector 515 is subsequently received by the prediction engine 110, and particularly the processor 200 forming a neural network system. The prediction engine 110 generates a probability score 520 of a future event 525 associated with the temporal record 510. For example, the probability score 520 may be a decimal value between 0 and 1, or a percentage in the range of 0-100% indicating the probability that the future event 525 will occur. Alternately, the probability score 520 may be a segmented value, such as the values low, medium, or high indicating the probability that the future event 525 will occur. Specifically, the prediction engine 110 generates the probability score 520 based on the temporal record and further temporal records. In the present example, the future event 525 may be a medically-related episode such as a cancer diagnosis, a cardiac arrest, or the like, or the future event 525 may be insurance related information, such as approval of a claim or claim amount. Accordingly, the prediction engine 110 may generate the probability score 520, for example based on comparing the medical and insurance history encoded in the vector 515 with the medical and insurance histories represented by the further temporal records.

In the present example, the probability score 520 is subsequently received by the vectorization engine 120. The vectorization engine generates a modified actor vector 530 which encodes event information associated with actual events from the temporal record, as well as predictive information based on the probability score 520 generated by the prediction engine 110.

The modified actor vector 530 is received, for example, by a grouping engine 535. The grouping engine 535 may be, for example, a processor associated with an output terminal. In other implementations, the grouping engine may be the same component as the prediction engine 110 or the vectorization engine 120. The grouping engine groups, at block 325 (shown in FIG. 3), the modified actor vector 530 with one or more additional vectors to form a group 540. Group 540, therefore, includes grouped vectors 542-1, 542-2, . . . , 542-m (collectively referred to as grouped vectors 542 and generically referred to as a grouped vector 542). In the present example, the grouped vector 542-2 corresponds with the modified actor vector 530. The group 540 may represent, for example, actors having a high risk of a cardiac-related medical episode. In another example, the group 540 may represent actors having a high probability of a claim amount above a predetermined threshold amount.

The group 540 is then output, for example, by displaying a listing of the actors corresponding to the grouped vectors 542 at an output terminal, according to block 330 of the method 300. In other implementations, the output terminal may be configured to display a visual representation of the group, or send the group or group information to a printer, another device, or network, or the like.

In other implementations, the probability scores or set of probability scores for future events associated with the temporal records may be output in addition to, or separate from the group. In further implementations, the group may be stored in the memory 204, the memory 254 or the repository 130 without outputting the group.

In other examples, the actor 505 may be a machine, and the temporal record 510 may be a maintenance and operational record. Accordingly, the events 512 may correspond to registered trouble tickets, or machine malfunction instances. The prediction engine 110 may generate a probability score 520 to predict future performance, breakdowns, required maintenance, and the machines may be grouped for example by type of maintenance needed, scheduling of maintenance, and the like. Thus, the system 100 may allow an operator to design a maintenance schedule and budget for future maintenance and repairs of the machine.

The present specification provides systems and methods of identifying groups of actors based on temporal records and outputting the groups of vectorized temporal records associated with the actors. In particular, the present specification provides a method of predicting events based on past events in the temporal records, similar events of other temporal records, or related events of other temporal records. In some implementations, the method further generates predictive vectors which relate actual and predicted events to identify the groups based on the probability score of future events, and the original temporal records. By employing predictive vectors, efficient clustering algorithms may be used to group the actors in consideration of both actual and predicted events.

Further, by vectorizing temporal records, the high dimensionality of the events, the event information, variable actor information, and so on, is reduced while maintaining and encoding the information. The reduced dimensionality therefore allows for increased the efficiency and speed of the prediction engine in processing the temporal records and generating probability scores.

The scope of the claims should not be limited by the implementations set forth in the above examples, but should be given the broadest interpretation consistent with the description and drawings as a whole.

1. A method comprising:
receiving, at a prediction engine, a vector representing a temporal record comprising an event having a timestamp;
generating, at the prediction engine, a probability score of a future event associated with the temporal record;
grouping the vector with one or more additional vectors to form a group, the grouping based on the temporal record and the probability score; and
outputting the group.

2. The method of claim 1, further comprising receiving, at the prediction engine, one or more further vectors representing corresponding further temporal records, each further temporal record comprising one or more events having corresponding timestamps.

3. The method of claim 2, wherein the probability score is generated based on the temporal record and the one or more further temporal records.

4. The method of claim 3, wherein the prediction engine comprises a neural network trained using the one or more further temporal records.

5. The method of claim 1, further comprising generating, at a vectorization engine, the vector based on the temporal record, the temporal record associated with an actor.

6. The method of claim 1, further comprising updating the vector based on an updated temporal record comprising the event having the timestamp and a further event having a further timestamp.

7. The method of claim 1, further comprising detecting a change in the temporal record, and updating the vector to reflect the change in the temporal record.

8. The method of claim 1, further comprising generating, at the prediction engine, further probability scores of a plurality of further future events associated with the temporal record.

9. The method of claim 8, wherein the grouping is based on the temporal record, the probability score and the further probability scores.

10. The method of claim 1, wherein outputting the group comprises generating a visual representation of the group.

11. The method of claim 1, further comprising:
comparing an actual event associated with the temporal record with a predicted event, the predicted event comprising the future event when the probability score is above a threshold score; and
generating a notification if an inconsistency is detected between the actual event and the predicted event.

12. A system comprising:
a prediction engine comprising a processor in communication with a computer-readable memory, the prediction engine to:
receive a vector representing a temporal record comprising an event having a timestamp;
generate a probability score of a future event associated with the temporal record; and
output a group comprising the vector and one or more additional vectors grouped together based on the temporal record and the probability score.

13. The system of claim 12, wherein the prediction engine is further configured to group the vector and the one or more additional vectors based on the temporal record and the probability score.

14. The system of claim 12, wherein the prediction engine is further configured to receive one or more further vectors representing corresponding further temporal records, each further temporal record comprising one or more events having corresponding timestamps.

15. The system of claim 14, wherein the prediction engine is configured to generate the probability score based on the temporal record and the one or more further temporal records.

16. The system of claim 15, wherein the prediction engine comprises a neural network trained using the one or more further temporal records.

17. The system of claim 12, further comprising a vectorization engine configured to generate the vector based on the temporal record, the temporal record associated with an actor.

18. The system of claim 17, wherein the vectorization engine is further configured to update the vector based on an updated temporal record comprising the event having the timestamp and a further event having a further timestamp.

19. The system of claim 17, wherein the vectorization engine is further configured to detect a change in the temporal record and update the vector to reflect the change in the temporal record.

20. The system of claim 12, wherein the prediction engine is further configured to generate probability scores of a plurality of future events associated with the temporal record.

21. The system of claim 20, wherein the prediction engine is configured to group the vector and the one or more additional vectors based on the temporal record, the probability score, and the further probability scores.

22. The system of claim 12, wherein the prediction engine is configured to generate a visual representation of the group.

23. The system of claim 12, wherein the prediction engine is further configured to:
compare an actual event associated with the temporal record with a predicted event, the predicted event comprising the future event when the probability score is above a threshold score; and
generating a notification if an inconsistency is detected between the actual event and the predicted event.

24. A non-transitory computer-readable storage medium having recorded thereon instructions executable by a processor of a prediction engine, the instructions to cause the processor to:
receive a vector representing a temporal record comprising an event having a timestamp, the temporal record associated with an actor;
generate a probability score of a future event associated with the temporal record;
group the vector with one or more additional vectors to form a group, the grouping based on the temporal record and the probability score; and
output the group.
